(19) United States
(12) Patent Application Publication
(10) Pub. No.: US 2018/0055675 A1
(43) Pub. Date: Mar. 1, 2018

(54) NECK BRACE

(71) Applicant: Melanie Deborah Hatto, Cape Coral, FL (US)

(72) Inventor: Melanie Deborah Hatto, Cape Coral, FL (US)

(21) Appl. No.: 15/249,807

(22) Filed: Aug. 29, 2016

(51) Int. Cl. A61F 5/055

(52) U.S. Cl. CPC A61F 5/055

(57) ABSTRACT

A neck brace includes a brace assembly and a strap assembly. A brace assembly can include a continuous main body configured to wrap around a wearer's neck. The main body can have first and second main body ends, left and right side portions, a chin portion, and a rear portion. A strap assembly includes a continuous lateral strap configured to laterally wrap around the wearer's head, and a longitudinal strap having first and second strap ends, the longitudinal strap being attached to the lateral strap at respective first and second connection points. The first and second main body ends are configured to connect to each other to secure the main body around the wearer's neck, and the first and second strap ends are configured to respectively attach to the first and second side portions.

### FIELD OF THE INVENTION

The present invention is related to medical devices, and more particularly, to a neck brace.

### BACKGROUND OF THE INVENTION

Within the medical and physical therapy fields, neck braces have been used to immobilize a wearer's neck due to various types of injuries to assist with healing processes.

Developments in the neck brace art have produced neck braces having increased complexities and cost.

### SUMMARY OF THE INVENTION

The present invention provides a neck brace that can immobilize a wearer's neck for rehabilitative purposes.

In an exemplary embodiment of the present invention, a neck brace can include a a brace assembly having a continuous main body configured to wrap around a wearer's neck, with the main body having first and second main body ends, left and right side portions, a chin portion, a rear portion, and one or more of an optional chin insert and an optional rear insert.

Such a neck brace can further include a strap assembly that includes a continuous lateral strap configured to wrap around the wearer's head, and a longitudinal strap having first and second strap ends, with the longitudinal strap being attached to the lateral strap at respective first and second connection points.

In exemplary aspects of the present invention, the first and second main body ends can be configured to connect to each other to secure the main body around a wearer's neck, and the first and second strap ends can be configured to respectively attach to the first and second side portions.

In another exemplary aspect, the first and second main body ends can be configured to connect to each other to secure the main body around the wearer's neck.

In a further exemplary aspect, the first and second strap ends can be configured to respectively attach to the first and second side portions, which secures the brace assembly and strap assembly together.

In yet another exemplary aspect, optional chin and/or neck inserts can be removably attached to the main body.

These and other exemplary aspects are further illustrated herein.

### BRIEF DESCRIPTION OF THE DRAWINGS

FIG. 1 illustrates an exploded view of an exemplary embodiment of a neck brace having a brace assembly and a strap assembly.

FIG. 2 illustrates an application of an exemplary neck brace with optional chin and rear inserts.

FIG. 3 illustrates another perspective of an application of an exemplary neck brace with optional chin and rear inserts.

### DETAILED DESCRIPTION

An object of the present invention is to provide a neck brace.

Another object of the present invention is to provide a neck brace that assists in immobilizing a user's neck.

These and other objects are provided via the illustrative embodiments and aspects described herein.

It should be noted that this disclosure includes a plurality of embodiments, each having a plurality of elements and/or aspects, and such elements and/or aspects need not necessarily be interpreted as being conjunctively required by one or more embodiments of the present invention. Rather, all combinations of all elements and/or aspects described herein can enable a separate embodiment of the present invention, which may be claimed with particularity in one or more future filed Non-Provisional Patent Applications. Moreover, any particular structure, arrangement, and/or functional logic disclosed herein, whether expressly or implicitly, are to be construed strictly as illustrative and enabling, and not necessarily limiting. Therefore, it is expressly set forth that such structure, arrangement, and functional logic, independently or in any combination of one of more thereof, are merely illustratively representative of one or more elements and/or aspects of one or more embodiments of the present invention and are not to be construed as necessary in a strict sense.

Further, to the extent the same element or aspect is defined differently anywhere within this disclosure, whether expressly or implicitly, the broader definition is to take absolute precedence, with the distinctions encompassed by the narrower definition to be strictly construed as optional.

Illustratively, perceived benefits of the present invention can include functional utility, whether expressly or implicitly stated herein, or apparent herefrom. However, it is expressly set forth that these benefits are not intended as exclusive. Therefore, any explicit, implicit, or apparent benefit from the disclosure herein is expressly deemed as applicable to the present invention.

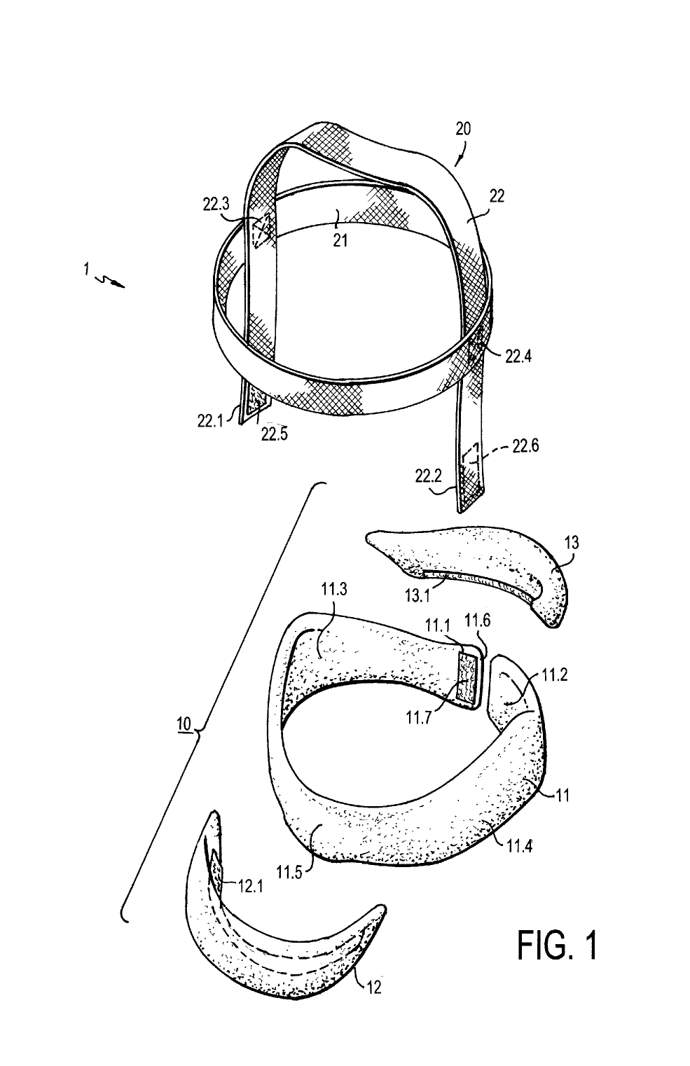

FIG. 1 illustrates an exemplary neck brace according to the present invention, in which a neck brace 1 can include a brace assembly 10 having a continuous main body 11 configured to wrap around a wearer's neck, with the main body having first and second main body ends 11.1, 11.2, left and right side portions 11.3, 11.4, a chin portion 11.5, and a rear portion 11.6. In another exemplary aspect, first and second main body ends 11.1, 11.2 can be connected to each other so as to place main body 11 in a continuous configuration for positioning around and against a wearer's neck. Notably, as illustrated, first main body end 11.1 can be provided with an attachment element, such as a hook element 11.7 (e.g., a VELCO hook element), to attach to second main body end 11.2, which can be formed of a loop-structured material or can be provided as a separate loop structure, such as a corresponding VELCO loop element. In another exemplary aspect, with main body 11 positioned around a wearer's neck, left and right side portions 11.3, 11.4 can be respectively positioned to the left and right sides of a wearer, chin portion 11.5 can be positioned below a wearer's chin, and rear portion 11.6 can be positioned against the rear of a wearer's neck.

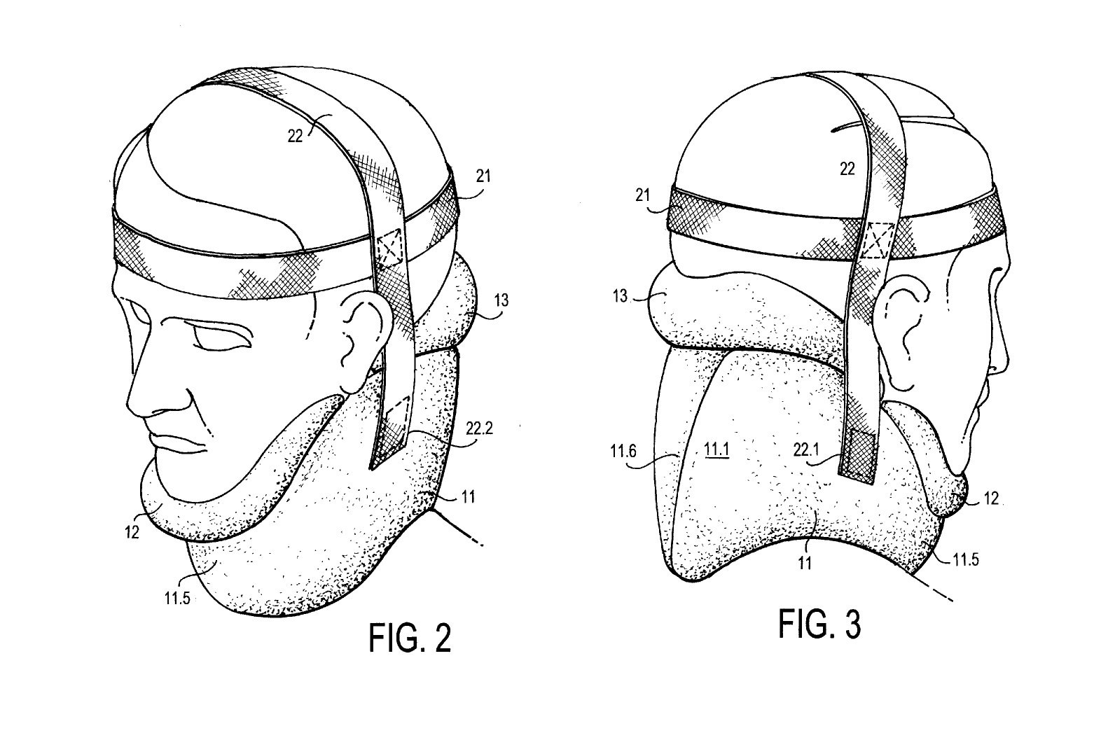

As further illustrated in FIG. 1, brace assembly 10 can further include an optional chin insert 12 and/or an optional rear insert 13, which are further described herein and illustrated in FIGS. 2 and 3.

As also illustrated in FIG. 1, neck brace 1 can further include a strap assembly 20 having a lateral strap 21 configured to laterally wrap around a wearer's head, and a longitudinal strap 22 having first and second strap ends 22.1, 22.2. Further, longitudinal strap 22 can be permanently or removably attached to lateral strap 21 at respective first and second attachment points 22.3, 22.4, which can be effectuated with any desired attachment element, such as with stitches (as illustratively shown), an adhesive, hook and loop, button, clasp, etc. Notably, as illustrated, upon attachment, lateral and longitudinal straps 21, 22 can be generally oriented in a perpendicular relationship to each other, however, such relative orientation can deviate therefrom and at any relative angle insofar as functionally compatible with the present invention as described.

In a further exemplary aspect, first and second strap ends 22.1, 22.2 can be configured to respectively attach to left and right side portions 11.3, 11.4, which can attach brace assembly 10 to strap assembly 20. For example, and not in limitation, such an attachment can be effectuated by providing strap ends 22.1, 22.2 with attachment elements, such as hook structures 22.5, 22.6, which can attachably engage left and right side portions 11.3, 11.4, which can be formed of a material having loop properties or provided with separate loop structures to the extent desired.

FIGS. 2 and 3 illustrate perspectives of an exemplary neck brace 1 worn by a user. As illustrated in FIG. 2, optional chin insert 12 can be removably attached to chin portion 11.5 of main body 11, and positioned between a user's chin and the chin portion, which can provide a wearer with additional frontal support when so desired. In an exemplary aspect, chin insert 12 can be provided with a hook element 12.1, which can engage chin portion 11.5 of main body 11. Further, chin insert 12 can be selectively utilized or removed when desired, such as when a wearer is sleeping, consuming, imbibing, or engaging in dental hygiene activities, for example and not in limitation.

As illustrated in FIG. 3, optional rear insert 13 can be removably attached to a rear portion 11.6 of main body 11, and be positioned between a user's head and the rear portion, so as to provide additional rear support when so desired. In an exemplary aspect, rear insert 13 can also be provided with a hook element 13.1 (or “hooking structure”), which can engage rear portion 11.6 of main body 11. And as with chin insert 12, rear insert 13 can be selectively utilized or removed to the extent desired.

According to the present invention, main body 11, optional chin insert 12, optional rear insert 13, latitudinal strap 21, and longitudinal strap 22 can be provided with any desired material or materials, including but not limited to one or more of a plastic, rubber, metal, wood, cloth, mesh, foam, naturally-occurring, and man-made material. In an exemplary aspect, one or more of main body 11, optional chin insert 12, and optional rear insert 13 can be provided with a compressible foam material with a cloth covering; whilst lateral and longitudinal straps 21, 22 can be provided with an elastomeric material.

It should be noted that particular attachment elements (also known as “connection elements”) have been described herein, however, the present invention is not limited thereto, and expressly contemplates as equivalents any known or apparent permanent or removable attachment element or elements insofar as functionally compatible with the present invention. For example and not in limitation, such an attachment element(s) can include one or more of a hook, loop, snap, catch, button, hole, magnet, ferromagnetic element, stitch, staple, pin, catch, adhesive, or any other functionally compatible structure or structures. Further, where each of two portions of the invention include corresponding attachment elements, either element can be provided with either portion.

It will be apparent to one of ordinary skill in the art that the manner of making and using the claimed invention has been adequately disclosed in the above-written description of the exemplary embodiments, aspects, and steps of the present invention.

It should be understood, however, that the invention is not necessarily limited to the specific embodiments, aspects, arrangement, and components shown and described above, but may be susceptible to numerous variations within the scope of the invention, with such variations deemed within the spirit of the present invention.

Therefore, the specification and drawings are to be regarded in an illustrative and enabling, rather than a restrictive, sense.

Accordingly, it will be understood that the above description of the embodiments of the present invention are susceptible to various modifications, changes, and adaptations, and the same are intended to be comprehended within the meaning and range of equivalents apparent to one of ordinary skill in the art.

Therefore I claim:

1. A neck brace, comprising:
a brace assembly including a continuous main body configured to wrap around a wearer's neck, with the main body having a first and second main body ends, left and right side portions, a chin portion, and a rear portion; and
a strap assembly including a continuous lateral strap configured to laterally wrap around the wearer's head, and a longitudinal strap having first and second strap ends, the longitudinal strap being attached to the lateral strap at respective first and second connection points;
wherein the first and second main body ends are configured to connect to each other to secure the main body around the wearer's neck, and the first and second strap ends are configured to respectively attach to the first and second side portions.

2. The neck brace of claim 1, wherein the brace assembly further includes a chin insert removably attached to the chin portion of the main body.

3. The neck brace of claim 2, wherein the chin insert includes an attachment element.

4. The neck brace of claim 3, wherein the attachment element is a hook element.

5. The neck brace of claim 1, wherein the brace assembly further includes a rear insert removably attached to the rear portion of the main body.

6. The neck brace of claim 5, wherein the rear insert includes a connection element.

7. The neck brace of claim 6, wherein the attachment element is a hooking structure.

8. The neck brace of claim 5, wherein the brace assembly further includes a chin insert removably attached to the chin portion of the main body.

9. The neck brace of claim 8, wherein the chin insert includes an attachment element.

10. The neck brace of claim 9, wherein the connection element is a hook element.
